(19) United States
(12) Patent Application Publication
(10) Pub. No.: US 2025/0303156 A1
(43) Pub. Date: Oct. 2, 2025

(54) CONCOMITANT TREATMENT OF TTFIELDS AND HMG-CO A REDUCTASE INHIBITORS

(71) Applicant: NOVOCURE GMBH, Baar (CH)

(72) Inventors: GITIT LAVY-SHAHAF, Haifa (IL); TALI VOLOSHIN-SELA, Haifa (IL); LILACH AVIGDOR, Haifa (IL)

(21) Appl. No.: 19/093,778

(22) Filed: Mar. 28, 2025

Related U.S. Application Data
(60) Provisional application No. 63/571,723, filed on Mar. 29, 2024.
(60) Provisional application No. 63/715,081, filed on Nov. 1, 2024.

(51) Int. Cl. A61N 1/36; A61K 31/22; A61K 31/337; A61K 31/366; A61K 31/40; A61K 31/405; A61K 31/47; A61K 31/505; A61P 35/00

(52) U.S. Cl. CPC A61N 1/36002; A61K 31/22; A61K 31/337; A61K 31/366; A61K 31/40; A61K 31/405; A61K 31/47; A61K 31/505; A61P 35/00

(57) ABSTRACT

Disclosed are methods of treating a subject in need thereof comprising applying alternating electric fields, at a frequency for a period of time, to a target site of the subject in need thereof; and administering one or more cholesterol lowering drugs to the subject in need thereof. Disclosed are methods of increasing survival of a subject comprising applying alternating electric fields, at a frequency for a period of time, to a target site of the subject in need thereof; and administering one or more cholesterol lowering drugs to the subject in need thereof. Disclosed are methods of increasing longevity of treatment with alternating electric field in a subject comprising applying alternating electric fields, at a frequency for a period of time, to a target site of the subject in need thereof; and administering one or more cholesterol lowering drugs to the subject in need thereof. Disclosed are methods of increasing efficacy of alternating electric fields in a cell comprising applying alternating electric fields, at a frequency for a period of time, to a cell; and contacting one or more cholesterol lowering drugs to the cell, thereby increasing efficacy of the alternating electric fields.

### CROSS-REFERENCE TO RELATED APPLICATIONS

This application claims the benefit of U.S. Provisional Patent Application Nos. 63/571,723, filed Mar. 29, 2024 and 63/715,081, filed Nov. 1, 2024, each of which is incorporated by reference herein in its entirety.

### BACKGROUND

Tumor Treating Fields, or TTFields, are typically low intensity (e.g., 1-3 V/cm) alternating electric fields within the intermediate frequency range (100-300 kHz). TTFields can be delivered through non-invasive transducer arrays across the anatomical region of a tumor. TTFields have been established as an anti-mitotic cancer treatment modality because they interfere with proper micro-tubule assembly during metaphase and eventually destroy the cells during telophase, cytokinesis, or subsequent interphase. TTFields have been shown to not affect the viability of non-dividing normal cells, nerves, and muscles because of their low intensity. TTFields therapy is an approved mono-treatment for recurrent glioblastoma, and an approved combination therapy with chemotherapy for newly diagnosed glioblastoma and unresectable malignant pleural mesothelioma patients. These electric fields are induced non-invasively by transducer arrays (i.e., arrays of electrodes) placed directly on the patient's scalp. TTFields also appear to be beneficial for treating tumors in other parts of the body.

### BRIEF SUMMARY

Disclosed are methods of treating a subject in need thereof comprising applying alternating electric fields, at a frequency for a period of time, to a target site of the subject in need thereof; and administering one or more cholesterol lowering drugs to the subject in need thereof.

Disclosed are methods of increasing survival of a subject comprising applying alternating electric fields, at a frequency for a period of time, to a target site of the subject in need thereof; and administering one or more cholesterol lowering drugs to the subject in need thereof.

Disclosed are methods of increasing longevity of treatment with alternating electric field in a subject comprising applying alternating electric fields, at a frequency for a period of time, to a target site of the subject in need thereof; and administering one or more cholesterol lowering drugs to the subject in need thereof.

Disclosed are methods of increasing efficacy of alternating electric fields in a cell comprising applying alternating electric fields, at a frequency for a period of time, to a cell; and contacting one or more cholesterol lowering drugs to the cell, thereby increasing efficacy of the alternating electric fields in the cell.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A and 1B show unexpected results from a clinical study in non small cell lung cancer (NSCLC) indicating that using statins in addition to TTFields treatment (without being a part of the clinical protocol) is associated with better overall survival compared to patients not receiving statins. FIG. 1A shows overall survival for one treatment cycle which is one month of TTFields treatment with a Pvalue of 0.0019 and hazard ratio of 0.3 his implies that patients treated with statins have a 70% lower risk of death compared to those not receiving statins. FIG. 1B shows overall survival for more than one month cycle of TTFields treatment with a Pvalue of 0.0030 and hazard ratio of 0.358.

FIG. 2 shows an examination of genes that are correlated with familial hypercholesterolemia which are shown to be partially upregulated in RNA isolated from different cell lines following treatment with TTFields.

FIGS. 3A and 3B show results from a transcriptomic fingerprint of cancer cell response to Tumor Treating Fields. FIG. 3A is a GSEA based on the MsigDB hallmarks gene set. To show the most significant results in one figure we constructed a heatmap of the NES along with the False Discovery Rate (FDR). Only pathways common and significant for at least 5 datasets were included in the heatmap showing 48 hour treatment FIG. 3B is a analysis based on the Ingenuity Pathway Analysis (IPA) canonical pathways. The dot blot shows the z score along with the log BH p-value of the different pathways in the different datasets. Only pathways common and significant to at least 5 datasets were included, showing the 48 hour treatment datasets, arranged according to the hierarchical clustering of

FIG. 4 shows results from an in vivo RNA transcriptome experiment in hepatocellular carcinoma mice treated with TTFields which clearly indicates an upregulation on pathways of cholesterol biosynthesis.

FIG. 5 shows a patient survival in a combination treatment of Docetaxel plus TTFields plus Simvastatin compared to patients that were treated with Docetaxel plus Simvastatin.

FIG. 6 shows patient survival a combination treatment of Docetaxel plus TTFields plus Atorvastatin compared to patients that were treated with Docetaxel plus Atorvastatin.

FIG. 7 shows patient survival a combination treatment of Docetaxel plus TTFields plus Rosuvastatin compared to patients that were treated with Docetaxel plus Rosuvastatin.

FIG. 8 shows unexpected results from a clinical study in glioblastoma multiforme (GBM) showing overall survival of patients treated with statins from start of treatment of the trial indicating that using statins in addition to TTFields treatment (without being a part of the clinical protocol) is associated with better overall survival compared to patients not receiving statins.

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cholesterol lowering drug” includes a plurality of such cholesterol lowering drugs, reference to “the cholesterol lowering drug” is a reference to one or more cholesterol lowering drugs and equivalents thereof known to those skilled in the art, and so forth.

The word “of” as used herein means any one member of a particular list and also includes any combination of members of that list.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the lungs or liver. In some aspects, a cell or population of cells that can be a target site or a target cell include, but are not limited to, a cancer cell (e.g., a lung or liver cancer cell). In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g., a target site such as a cell). In some aspects, the alternating electrical field can be in a single direction or multiple directional. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and is described in U.S. Pat. No. 7,565,205, which is incorporated herein by reference in its entirety for its teaching of TTFields.

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. “Subject” can be used interchangeably with “individual” or “patient.” For example, the subject of administration can mean the recipient of the alternating electrical field. For example, the subject of administration can be a subject with ovarian cancer or lung cancer.

By “treat” is meant to administer or apply a therapeutic, such as alternating electric fields and a vector, to a subject, such as a human or other mammal (for example, an animal model), that has cancer or has an increased susceptibility for developing cancer, in order to prevent or delay a worsening of the effects of the disease or infection, or to partially or fully reverse the effects of cancer. For example, treating a subject having non-small cell lung cancer or hepatocellular carcinoma can comprise delivering a therapeutic to a cell in the subject.

By “prevent” is meant to minimize or decrease the chance that a subject develops cancer.

As used herein, the terms “administering” and “administration” refer to any method of providing a cholesterol lowering drug to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intratumor administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat cancer. In further various aspects, a preparation can be administered prophylactically; that is, administered for prevention of cancer. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 kHz and 1 MHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be about 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is in a range from about 200 kHz to about 400 kHz, from about 250 kHz to about 350 kHz, and may be around 300 kHz.

In some aspects, the field strength of the alternating electric fields can be between 0.5 and 4 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be about 1.75 V/cm RMS. In some embodiments the field strength is at least 1 V/cm RMS. In some aspects, the field strength can be about or at least 0.9 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

### C. Compositions

Disclosed are compositions and formulations comprising one or more cholesterol lowering drugs. In some embodiments the formulation further includes a pharmaceutically acceptable carrier or diluent. For example, disclosed are pharmaceutical compositions, comprising a cholesterol lowering drug and a pharmaceutically acceptable carrier. For example, disclosed are pharmaceutical compositions, comprising Hydroxymethylglutaryl-CoA (HMG CoA) reductase inhibitors, and a pharmaceutically acceptable carrier. Disclosed also are pharmaceutical compositions, comprising a cholesterol lowering drug and a pharmaceutically acceptable diluent.

In some aspects, the one or more cholesterol lowering drugs is a hydroxymethylglutaryl-CoA (HMG CoA) reductase inhibitor, bempedoic acid, a fibrate, a resin, an apolipoprotein B synthesis inhibitor, a microsomal transfer protein inhibitor, PCSK9 inhibitors (Alirocumab (Praluent), Evolocumab (Repatha), Leqvio, LDL-C siRNA, L-IFTA+, BMS-962476, Pep2-8, Annexin A2, Pseurotin 1, Silibinin A, MK 0616, BMS 8444421, ISIS 394814, and SPC4061). In some aspects, the one or more cholesterol lowering drugs is a fibric acid derivatives or fibrates (e.g. Tricor, Lopid, Trilipix, Lipofen, Antara, Riglide, Lofibra, Fibricor and Fenoglide), a cholesterol absorption inhibitor (e.g. Ezetimibe, (SCH 58235) and SCH-48461), Omega-3 fatty acids, or a citrate lyase inhibitor.

In some aspects, the HMG CoA reductase inhibitor is selected from atorvastatin (LIPITOR), fluvastatin (LESCOL XL), lovastatin (ALTOPREV), pitavastatin (LIVALO), pravastatin (Pravachol), rosuvastatin (CRESTOR), Rosuvastatin (Crestor), and simvastatin (ZOCOR).

In some aspects, the cholesterol lowering drug can be administered with a pharmaceutically acceptable carrier and/or diluent in any of the disclosed methods.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidylcholine (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. In the methods described herein, delivery of the disclosed compositions to cells can be via a variety of mechanisms. The pharmaceutical composition may be administered in a number of ways depending on whether local or systemic treatment is desired, and on the area to be treated.

### 1. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### D. Methods of Treating

The inventors have discovered that cancer patients who receive TTFields and a cholesterol-lowering drug (e.g., a HMG-CoA reductase inhibitor) surprisingly have a better overall survival than patients who receive a cholesterol-lowering drug (e.g., a HMG-CoA reductase inhibitor). Accordingly, disclosed are methods of treating a subject in need thereof comprising applying alternating electric fields to a target site of the subject in need thereof; and administering one or more cholesterol lowering drugs to the subject in need thereof.

In some aspects, the one or more cholesterol lowering drugs is a hydroxymethylglutaryl-CoA (HMG CoA) reductase inhibitor, bempedoic acid, a fibrate, a resin, an apolipoprotein B synthesis inhibitor, a microsomal transfer protein inhibitor, PCSK9 inhibitors (Alirocumab (Praluent), Evolocumab (Repatha), Leqvio, LDL-C siRNA, L-IFTA+, BMS-962476, Pep2-8, Annexin A2, Pseurotin 1, Silibinin A, MK 0616, BMS 8444421, ISIS 394814, and SPC4061). In some aspects, the one or more cholesterol lowering drugs is a fibric acid derivatives or fibrates (e.g. Tricor, Lopid, Trilipix, Lipofen, Antara, Riglide, Lofibra, Fibricor and Fenoglide), a cholesterol absorption inhibitor (e.g. Ezetimibe, (SCH 58235) and SCH-48461), Omega-3 fatty acids, or a citrate lyase inhibitor.

In some aspects, the subject in need thereof has cancer. In some aspects, the subject in need thereof has mesothelioma, ovarian cancer, or lung cancer. In some aspects, the subject in need thereof has lung cancer (e.g., non-small cell lung cancer), hepatocellular cancer, brain cancer, pancreatic cancer, breast cancer, colon cancer, ovarian cancer, mesothelioma, renal cancer, or glioblastoma.

In some aspects, the subject has previously undergone chemotherapy. In some aspects, the subject is currently undergoing chemotherapy. In some aspects, the subject has previously failed chemotherapy.

In some aspects, the target site comprises one or more cancer cells. In some aspects, the target site comprises one or more lung cancer cells, mesothelioma cells, ovarian cancer cells, brain cancer cells, pancreatic cancer cells, breast cancer cells, hepatocellular cancer cells, or colon cancer cells.

In some aspects, the disclosed methods of treating further comprise detecting the presence of cholesterol and/or lipid biosynthesis in the subject. In some aspects, detecting the presence of cholesterol and/or lipid biosynthesis in the subject can occur before or after applying the alternating electric field and/or before or after administering one or more cholesterol lowering drugs. Thus, in some aspects, the disclosed methods comprise after applying the alternating electric field and before administering one or more cholesterol lowering drugs, detecting the presence of increased cholesterol and/or lipid biosynthesis in the subject. In some aspects, the disclosed methods comprise, prior to applying the alternating electric field, detecting the presence of increased cholesterol levels and/or increased lipid biosynthesis in the subject. In some aspects, detecting the presence and/or an increase of cholesterol and/or lipid biosynthesis allows for a comparison to a baseline or reference point of cholesterol and/or lipid biosynthesis. In some aspects, a baseline or reference point for cholesterol and/or lipid biosynthesis can be a range found prior to any treatment. In some aspects, a baseline or reference point for cholesterol and/or lipid biosynthesis can be a range found in healthy subjects. In some aspects, a healthy subject is a subject that does not have cancer. In some aspects, a healthy subject is a subject with no known infections, diseases or disorders. In some aspects, a healthy subject is a subject with no known medical conditions.

In some aspects, the disclosed methods of treating further comprise detecting the presence of upregulation of genes correlated with familial hypercholesterolemia in the subject. In some aspects, detecting the presence of upregulation of genes correlated with familial hypercholesterolemia in the subject can occur before or after applying the alternating electric field and/or before or after administering one or more cholesterol lowering drugs. Thus, in some aspects, the disclosed methods comprise after applying the alternating electric field and before administering one or more cholesterol lowering drugs, detecting the presence of upregulation of genes correlated with familial hypercholesterolemia in the subject. In some aspects, the disclosed methods comprise, prior to applying the alternating electric field, detecting the presence of upregulation of genes correlated with familial hypercholesterolemia in the subject. In some aspects, detecting the upregulation of genes correlated with familial hypercholesterolemia allows for a comparison to a baseline or reference point of expression of genes correlated with familial hypercholesterolemia. In some aspects, a baseline or reference point for expression of genes correlated with familial hypercholesterolemia can be a range found prior to any treatment. In some aspects, a baseline or reference point for expression of genes correlated with familial hypercholesterolemia can be a range found in healthy subjects. In some aspects, a healthy subject is a subject that does not have cancer. In some aspects, a healthy subject is a subject with no known infections, diseases or disorders. In some aspects, a healthy subject is a subject with no known medical condition.

In some aspects, the alternating electric fields are applied before, after, or simultaneously with administering the one or more cholesterol lowering drugs. In some aspects, the step of applying the alternating electric fields begins at least one hour before administering a cholesterol lowering drug. In some aspects, the step of applying the alternating electric fields begins at least 30 minutes before administering a cholesterol lowering drug. In some aspects, applying the alternating electric fields and administering a cholesterol lowering drug simultaneously can mean applying the alternating electric fields within 5, 10, 15, 20, 25, 30, 35, 40, 45, 50, 55, or 60 minutes before or after administering a cholesterol lowering drug. In some aspects, the alternating electric fields can be applied and the cholesterol lowering drug administered at least 1, 2, 3, 4, 5, 6, 7, 8, 9, 10, 11, 12, 13, 14, 15, 16, 17, 18, 19, 20, 21, 22, 23, or 24 hours from each other.

In some aspects, the disclosed methods of treating can further comprise administering a cancer therapeutic. In some aspects, the cancer therapeutic is a known cancer therapeutic other than cholesterol lowering drugs. For example, the cancer therapeutic can be, but is not limited to, chemotherapeutic agent, radiation, immunotherapy, TKI inhibitors, mTOR inhibitors, Akt inhibitors, PI3K inhibitors, PARP inhibitors, VEGF inhibitors, FGF inhibitors, aromatase inhibitors, biologics, anti-LAG3 agents, a hormone therapy as well as any combinations thereof. In some aspects, the chemotherapeutic agent can be, but is not limited to, docetaxel, carboplatin, cisplatin, oxaliplatin, Dacarbazine, procarbazine, temozolomide, Busulfan, Lomustine, bendamustine, cyclophosphamide, ifosfamide, Fluorouracil, Gemcitabine, Doxorubicin, Methotrexate, Capecitabine, Altretamine, Epirubicin, Irinotecan, Daunorubicin, Paclitaxel, Chlorambucil, Idarubicin, Cabazitaxel, Floxuridine, Pembrolizumab, Nivolumab, durvalumab, Cemiplimab, Ipilimumab, atezolizumab, Niraparib, Olaparib, Bevacizumab, alpelisib, Nab-paclitaxel (Abraxane), Sacituzumab govitecan, Dato-Dxd, taxol, Lenvatinib, everolimus, Letrozole, Gefitinib, Erlotinib, or sorafenib.

In some aspects of the disclosed methods of treating, there is a reduction in cancer cell proliferation in the subject (e.g., less spreading of the cancer cells in the subject). In some aspects, cancer cells in the subject can be killed. In some aspects, the reduction in cancer cell proliferation or the cancer cell death is increased compared to subjects treated with docetaxel and statin alone (e.g., no alternating electric fields). In some aspects, the reduction in cancer cell proliferation or the cancer cell death is increased in a subject with non-small cell lung cancer compared to subjects with non-small cell lung cancer treated with docetaxel and statin alone (e.g., no alternating electric fields).

In some aspects, the one or more cholesterol lowering drugs are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g., computed tomography-guided, during surgery or biopsy) or via inhalation.

In some aspects, the frequency of the alternating electric fields is between 50 kHz and 1 MHz. In some aspects, the frequency of the alternating electric field is 150 or 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of any of those described herein.

### E. Methods of Increasing Survival

Disclosed are methods of increasing survival of a subject comprising applying alternating electric fields, at a frequency for a period of time, to a target site of the subject in need thereof; and administering one or more cholesterol lowering drugs to the subject in need thereof.

In some aspects, the one or more cholesterol lowering drugs is a hydroxymethylglutaryl-CoA (HMG CoA) reductase inhibitor, bempedoic acid, a fibrate, a resin, an apolipoprotein B synthesis inhibitor, a microsomal transfer protein inhibitor, PCSK9 inhibitors (Alirocumab (Praluent), Evolocumab (Repatha), Leqvio, LDL-C siRNA, L-IFTA+, BMS-962476, Pep2-8, Annexin A2, Pseurotin 1, Silibinin A, MK 0616, BMS 8444421, ISIS 394814, and SPC4061). In some aspects, the one or more cholesterol lowering drugs is a fibric acid derivatives or fibrates (e.g. Tricor, Lopid, Trilipix, Lipofen, Antara, Riglide, Lofibra, Fibricor and Fenoglide), a cholesterol absorption inhibitor (e.g. Ezetimibe, (SCH 58235) and SCH-48461), Omega-3 fatty acids, or a citrate lyase inhibitor.

In some aspects, the subject in need thereof has cancer. In some aspects, the subject in need thereof has mesothelioma, ovarian cancer, or lung cancer. In some aspects, the subject in need thereof has lung cancer (e.g., non-small cell lung cancer), hepatocellular cancer, brain cancer, pancreatic cancer, breast cancer, colon cancer, ovarian cancer, mesothelioma, renal cancer, or glioblastoma.

In some aspects, the subject has previously undergone chemotherapy. In some aspects, the subject is currently undergoing chemotherapy. In some aspects, the subject has previously failed chemotherapy.

In some aspects, the target site comprises one or more cancer cells. In some aspects, the target site comprises one or more lung cancer cells, mesothelioma cells, ovarian cancer cells, brain cancer cells, pancreatic cancer cells, breast cancer cells, hepatocellular cancer cells, renal cancer cells, glioblastoma cancer cells, or colon cancer cells.

In some aspects, the disclosed methods of increasing survival further comprise detecting the presence of cholesterol and/or lipid biosynthesis in the subject. In some aspects, detecting the presence of cholesterol and/or lipid biosynthesis in the subject can occur before or after applying the alternating electric field and/or before or after administering one or more cholesterol lowering drugs. Thus, in some aspects, the disclosed methods comprise after applying the alternating electric field and before administering one or more cholesterol lowering drugs, detecting the presence of increased cholesterol and/or lipid biosynthesis in the subject. In some aspects, the disclosed methods comprise, prior to applying the alternating electric field, detecting the presence of increased cholesterol and/or lipid biosynthesis in the subject. In some aspects, detecting the presence and/or an increase of cholesterol and/or lipid biosynthesis allows for a comparison to a baseline or reference point of cholesterol and/or lipid biosynthesis. In some aspects, a baseline or reference point for cholesterol and/or lipid biosynthesis can be a range found prior to any treatment. In some aspects, a baseline or reference point for cholesterol and/or lipid biosynthesis can be a range found in healthy subjects. In some aspects, a healthy subject is a subject that does not have cancer. In some aspects, a healthy subject is a subject with no known infections, diseases or disorders. In some aspects, a healthy subject is a subject with no known medical condition.

In some aspects, the disclosed methods of increasing survival further comprise detecting the presence of upregulation of genes correlated with familial hypercholesterolemia in the subject. In some aspects, detecting the presence of upregulation of genes correlated with familial hypercholesterolemia in the subject can occur before or after applying the alternating electric field and/or before or after administering one or more cholesterol lowering drugs. Thus, in some aspects, the disclosed methods comprise after applying the alternating electric field and before administering one or more cholesterol lowering drugs, detecting the presence of upregulation of genes correlated with familial hypercholesterolemia in the subject. In some aspects, the disclosed methods comprise, prior to applying the alternating electric field, detecting the presence of upregulation of genes correlated with familial hypercholesterolemia in the subject. In some aspects, detecting the upregulation of genes correlated with familial hypercholesterolemia allows for a comparison to a baseline or reference point of expression of genes correlated with familial hypercholesterolemia. In some aspects, a baseline or reference point for expression of genes correlated with familial hypercholesterolemia can be a range found prior to any treatment. In some aspects, a baseline or reference point for expression of genes correlated with familial hypercholesterolemia can be a range found in healthy subjects. In some aspects, a healthy subject is a subject that does not have cancer. In some aspects, a healthy subject is a subject with no known infections, diseases or disorders. In some aspects, a healthy subject is a subject with no known medical conditions.

In some aspects, the alternating electric fields are applied before, after, or simultaneously with administering the one or more cholesterol lowering drugs. In some aspects, the step of applying the alternating electric fields begins at least one hour before administering a cholesterol lowering drug. In some aspects, the step of applying the alternating electric fields begins at least 30 minutes before administering a cholesterol lowering drug. In some aspects, applying the alternating electric fields simultaneously with administering a cholesterol lowering drug can mean applying the alternating electric fields within 5, 10, 15, 20, 25, 30, 35, 40, 45, 50, 55, or 60 minutes before or after administering a cholesterol lowering drug. In some aspects, the alternating electric fields can be applied and the cholesterol lowering drug administered at least 1, 2, 3, 4, 5, 6, 7, 8, 9, 10, 11, 12, 13, 14, 15, 16, 17, 18, 19, 20, 21, 22, 23, or 24 hours from each other.

In some aspects, the disclosed methods of increasing survival can further comprise administering a cancer therapeutic. In some aspects, the cancer therapeutic is a known cancer therapeutic other than cholesterol lowering drugs. For example, the cancer therapeutic can be, but is not limited to, chemotherapeutic agent, radiation, immunotherapy, or hormone therapy. In some aspects, the chemotherapeutic agent can be, but is not limited to, docetaxel, carboplatin, cisplatin, oxaliplatin, Dacarbazine, procarbazine, temozolamide, Busulfan, bendamustine, cyclophosphamide, ifosfamide, Fluorouracil, Gemcitabine, Doxorubicin, Methotrexate, Capecitabine, Altretamine, Epirubicin, Irinotecan, Daunorubicin, Paclitaxel, Chlorambucil, Idarubicin, Cabazitaxel, Floxuridine, Pembrolizumab, atezolizumab, alpelisib, gemcitabine, Nab-paclitaxel (Abraxane). Sacituzumab govitecan, Dato-Dxd, taxol, docetaxel, Gefitinib, or sorafenib.

In some aspects, the survival is increased in a subject with non-small cell lung cancer treated with TTFields, Docetaxel and statins compared to subjects with non-small cell lung cancer treated with docetaxel and statins alone (e.g., no alternating electric fields).

In some aspects, the one or more cholesterol lowering drugs are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g., computed tomography-guided, during surgery or biopsy) or via inhalation.

In some aspects, the frequency of the alternating electric fields is between 50 kHz and 1 MHz. In some aspects, the frequency of the alternating electric field is 150 or 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of any of those described herein.

### F. Methods of Increasing Longevity of Treatment with Alternating Electric Field

Disclosed are methods of increasing longevity of treatment with alternating electric field in a subject comprising applying alternating electric fields, at a frequency for a period of time, to a target site of the subject in need thereof; and administering one or more cholesterol lowering drugs to the subject in need thereof.

In some aspects, the one or more cholesterol lowering drugs is a hydroxymethylglutaryl-CoA (HMG CoA) reductase inhibitor, bempedoic acid, a fibrate, a resin, an apolipoprotein B synthesis inhibitor, a microsomal transfer protein inhibitor, PCSK9 inhibitors (Alirocumab (Praluent), Evolocumab (Repatha), Leqvio, LDL-C siRNA, L-IFTA+, BMS-962476, Pep2-8, Annexin A2, Pseurotin 1, Silibinin A, MK 0616, BMS 8444421, ISIS 394814, and SPC4061). In some aspects, the one or more cholesterol lowering drugs is a fibric acid derivatives or fibrates (e.g. Tricor, Lopid, Trilipix, Lipofen, Antara, Riglide, Lofibra, Fibricor and Fenoglide), a cholesterol absorption inhibitor (e.g. Ezetimibe, (SCH 58235) and SCH-48461), Omega-3 fatty acids, or a citrate lyase inhibitor.

In some aspects, the subject in need thereof has cancer. In some aspects, the subject in need thereof has mesothelioma, ovarian cancer, or lung cancer. In some aspects, the subject in need thereof has lung cancer (e.g., non-small cell lung cancer), hepatocellular cancer, brain cancer, pancreatic cancer, breast cancer, colon cancer, ovarian cancer, mesothelioma, renal cancer, or glioblastoma.

In some aspects, the subject has previously undergone chemotherapy. In some aspects, the subject is currently undergoing chemotherapy. In some aspects, the subject has previously failed chemotherapy.

In some aspects, the target site comprises one or more cancer cells. In some aspects, the target site comprises one or more lung cancer cells, mesothelioma cells, ovarian cancer cells, brain cancer cells, pancreatic cancer cells, breast cancer cells, hepatocellular cancer cells, or colon cancer cells.

In some aspects, the disclosed methods of increasing longevity of treatment with alternating electric field, further comprise detecting the presence of cholesterol and/or lipid biosynthesis in the subject. In some aspects, detecting the presence of cholesterol and/or lipid biosynthesis in the subject can occur before or after applying the alternating electric field and/or before or after administering one or more cholesterol lowering drugs. Thus, in some aspects, the disclosed methods comprise after applying the alternating electric field and before administering one or more cholesterol lowering drugs, detecting the presence of increased cholesterol and/or lipid biosynthesis in the subject. In some aspects, the disclosed methods comprise, prior to applying the alternating electric field, detecting the presence of increased cholesterol and/or lipid biosynthesis in the subject. In some aspects, detecting the presence and/or an increase of cholesterol and/or lipid biosynthesis allows for a comparison to a baseline or reference point of cholesterol and/or lipid biosynthesis. In some aspects, a baseline or reference point for cholesterol and/or lipid biosynthesis can be a range found prior to any treatment. In some aspects, a baseline or reference point for cholesterol and/or lipid biosynthesis can be a range found in healthy subjects. In some aspects, a healthy subject is a subject that does not have cancer. In some aspects, a healthy subject is a subject with no known infections, diseases or disorders. In some aspects, a healthy subject is a subject with no known medical condition.

In some aspects, the disclosed methods of increasing longevity of treatment with alternating electric field further comprise detecting the presence of upregulation of genes correlated with familial hypercholesterolemia in the subject. In some aspects, detecting the presence of upregulation of genes correlated with familial hypercholesterolemia in the subject can occur before or after applying the alternating electric field and/or before or after administering one or more cholesterol lowering drugs. Thus, in some aspects, the disclosed methods comprise after applying the alternating electric field and before administering one or more cholesterol lowering drugs, detecting the presence of upregulation of genes correlated with familial hypercholesterolemia in the subject. In some aspects, the disclosed methods comprise, prior to applying the alternating electric field, detecting the presence of upregulation of genes correlated with familial hypercholesterolemia in the subject. In some aspects, detecting the upregulation of genes correlated with familial hypercholesterolemia allows for a comparison to a baseline or reference point of expression of genes correlated with familial hypercholesterolemia. In some aspects, a baseline or reference point for expression of genes correlated with familial hypercholesterolemia can be a range found prior to any treatment. In some aspects, a baseline or reference point for expression of genes correlated with familial hypercholesterolemia can be a range found in healthy subjects. In some aspects, a healthy subject is a subject that does not have cancer. In some aspects, a healthy subject is a subject with no known infections, diseases or disorders. In some aspects, a healthy subject is a subject with no known medical conditions.

In some aspects, the alternating electric fields are applied before, after, or simultaneously with administering the one or more cholesterol lowering drugs. In some aspects, the step of applying the alternating electric fields begins at least one hour before administering a cholesterol lowering drug. In some aspects, the step of applying the alternating electric fields begins at least 30 minutes before administering a cholesterol lowering drug. In some aspects, applying the alternating electric fields simultaneously with administering a cholesterol lowering drug can mean applying the alternating electric fields within 5, 10, 15, 20, 25, 30, 35, 40, 45, 50, 55, or 60 minutes before or after administering a cholesterol lowering drug. In some aspects, the alternating electric fields can be applied and the cholesterol lowering drug administered at least 1, 2, 3, 4, 5, 6, 7, 8, 9, 10, 11, 12, 13, 14, 15, 16, 17, 18, 19, 20, 21, 22, 23, or 24 hrs from each other.

In some aspects, the disclosed methods of increasing longevity of treatment with alternating electric field can further comprise administering a cancer therapeutic. In some aspects, the cancer therapeutic is a known cancer therapeutic other than cholesterol lowering drugs. For example, the cancer therapeutic can be, but is not limited to, chemotherapeutic agent, radiation, immunotherapy, or hormone therapy. In some aspects, the chemotherapeutic agent can be, but is not limited to, docetaxel, carboplatin, cisplatin, oxaliplatin, Dacarbazine, procarbazine, temozolamide, Busulfan, bendamustine, cyclophosphamide, ifosfamide, Fluorouracil, Gemcitabine, Doxorubicin, Methotrexate, Capecitabine, Altretamine, Epirubicin, Irinotecan, Daunorubicin, Paclitaxel, Chlorambucil, Idarubicin, Cabazitaxel, Floxuridine, Pembrolizumab, atezolizumab, alpelisib, gemcitabine, Nab-paclitaxel (Abraxane), Sacituzumab govitecan, Dato-Dxd, taxol, docetaxel, Gefitinib, or sorafenib.

In some aspects, the longevity of treatment is increased in a subject with non-small cell lung cancer compared to subjects with non-small cell lung cancer treated with TTFields, Docetaxel and statins treated with docetaxel and statin alone (e.g., no alternating electric fields).

In some aspects, the one or more cholesterol lowering drugs are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g., computed tomography-guided, during surgery or biopsy) or via inhalation.

In some aspects, the frequency of the alternating electric fields is between 50 kHz and 1 MHz. In some aspects, the frequency of the alternating electric field is 150 or 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of any of those described herein.

### G. Methods of Increasing Efficacy of Alternating Electric Field

Disclosed are methods of increasing efficacy of alternating electric fields in a cell comprising applying alternating electric fields, at a frequency for a period of time, to a cell; and contacting one or more cholesterol lowering drugs to the cell, thereby increasing efficacy of the alternating electric fields.

In some aspects, the one or more cholesterol lowering drugs is a hydroxymethylglutaryl-CoA (HMG CoA) reductase inhibitor, bempedoic acid, a fibrate, a resin, an apolipoprotein B synthesis inhibitor, a microsomal transfer protein inhibitor, PCSK9 inhibitors (Alirocumab (Praluent), Evolocumab (Repatha), Leqvio, LDL-C siRNA, L-IFTA+, BMS-962476, Pep2-8, Annexin A2, Pseurotin 1, Silibinin A, MK 0616, BMS 8444421, ISIS 394814, and SPC4061). In some aspects, the one or more cholesterol lowering drugs is a fibric acid derivatives or fibrates (e.g. Tricor, Lopid, Trilipix, Lipofen, Antara, Riglide, Lofibra, Fibricor and Fenoglide), a cholesterol absorption inhibitor (e.g. Ezetimibe, (SCH 58235) and SCH-48461), Omega-3 fatty acids, or a citrate lyase inhibitor.

In some aspects, the cell is in culture (e.g., in vitro). In some aspects, the cell is in a subject (e.g., in vivo). In some aspects, the cell can be a cancer cell. In some aspects, the cancer cell can be, but is not limited to, lung cancer cells, mesothelioma cells, ovarian cancer cells, brain cancer cells, pancreatic cancer cells, breast cancer cells, hepatocellular cancer cells, or colon cancer cells.

In some aspects, when the method occurs in vivo, the subject has previously undergone chemotherapy. In some aspects, the subject is currently undergoing chemotherapy. In some aspects, the subject has previously failed chemotherapy.

In some aspects, the disclosed methods of increasing efficacy of alternating electric field further comprise detecting the presence of cholesterol and/or lipid biosynthesis in the subject. In some aspects, detecting the presence of cholesterol and/or lipid biosynthesis in the subject can occur before or after applying the alternating electric field and/or before or after administering one or more cholesterol lowering drugs. Thus, in some aspects, the disclosed methods comprise after applying the alternating electric field and before administering one or more cholesterol lowering drugs, detecting the presence of increased cholesterol and/or lipid biosynthesis in the subject. In some aspects, the disclosed methods comprise, prior to applying the alternating electric field, detecting the presence of increased cholesterol and/or lipid biosynthesis in the subject. In some aspects, detecting the presence and/or an increase of cholesterol and/or lipid biosynthesis allows for a comparison to a baseline or reference point of cholesterol and/or lipid biosynthesis. In some aspects, a baseline or reference point for cholesterol and/or lipid biosynthesis can be a range found prior to any treatment. In some aspects, a baseline or reference point for cholesterol and/or lipid biosynthesis can be a range found in healthy subjects. In some aspects, a healthy subject is a subject that does not have cancer. In some aspects, a healthy subject is a subject with no known infections, diseases or disorders. In some aspects, a healthy subject is a subject with no known medical conditions.

In some aspects, the disclosed methods of increasing efficacy of alternating electric field further comprise detecting the presence of upregulation of genes correlated with familial hypercholesterolemia in the subject. In some aspects, detecting the presence of upregulation of genes correlated with familial hypercholesterolemia in the subject can occur before or after applying the alternating electric field and/or before or after administering one or more cholesterol lowering drugs. Thus, in some aspects, the disclosed methods comprise after applying the alternating electric field and before administering one or more cholesterol lowering drugs, detecting the presence of upregulation of genes correlated with familial hypercholesterolemia in the subject. In some aspects, the disclosed methods comprise, prior to applying the alternating electric field, detecting the presence of upregulation of genes correlated with familial hypercholesterolemia in the subject. In some aspects, detecting the upregulation of genes correlated with familial hypercholesterolemia allows for a comparison to a baseline or reference point of expression of genes correlated with familial hypercholesterolemia. In some aspects, a baseline or reference point for expression of genes correlated with familial hypercholesterolemia can be a range found prior to any treatment. In some aspects, a baseline or reference point for expression of genes correlated with familial hypercholesterolemia can be a range found in healthy subjects. In some aspects, a healthy subject is a subject that does not have cancer. In some aspects, a healthy subject is a subject with no known infections, diseases or disorders. In some aspects, a healthy subject is a subject with no known medical conditions.

In some aspects, the alternating electric fields are applied before, after, or simultaneously with administering the one or more cholesterol lowering drugs. In some aspects, the step of applying the alternating electric fields begins at least one hour before administering a cholesterol lowering drug. In some aspects, the step of applying the alternating electric fields begins at least 30 minutes before administering a cholesterol lowering drug. In some aspects, applying the alternating electric fields and administering a cholesterol lowering drug simultaneously can mean applying the alternating electric fields within 5, 10, 15, 20, 25, 30, 35, 40, 45, 50, 55, or 60 minutes before or after administering a cholesterol lowering drug. In some aspects, the alternating electric fields can be applied and the cholesterol lowering drug administered at least 1, 2, 3, 4, 5, 6, 7, 8, 9, 10, 11, 12, 13, 14, 15, 16, 17, 18, 19, 20, 21, 22, 23, or 24 hrs from each other.

In some aspects, the disclosed methods of treating can further comprise administering a cancer therapeutic. In some aspects, the cancer therapeutic is a known cancer therapeutic other than cholesterol lowering drugs. For example, the cancer therapeutic can be, but is not limited to, chemotherapeutic agent, radiation, immunotherapy, or hormone therapy. In some aspects, the chemotherapeutic agent can be, but is not limited to, docetaxel, carboplatin, cisplatin, oxaliplatin, Dacarbazine, procarbazine, temozolamide, Busulfan, bendamustine, cyclophosphamide, ifosfamide, Fluorouracil, Gemcitabine, Doxorubicin, Methotrexate, Capecitabine, Altretamine, Epirubicin, Irinotecan, Daunorubicin, Paclitaxel, Chlorambucil, Idarubicin, Cabazitaxel, Floxuridine, Pembrolizumab, atezolizumab, alpelisib, gemcitabine, Nab-paclitaxel (Abraxane), Sacituzumab govitecan, Dato-Dxd, taxol, docetaxel, Gefitinib, or sorafenib.

In some aspects, the one or more cholesterol lowering drugs are administered intratumorally, intracranially, intraventricularly, intrathecally, epidurally, intradurally, intravascularly, intravenously (targeted or non-targeted), intraarterially, intramuscularly, subcutaneously, intraperitoneally, orally, intranasally, via intratumor injection (e.g., computed tomography-guided, during surgery or biopsy) or via inhalation.

In some aspects, the frequency of the alternating electric fields is between 50 kHz and 1 MHz. In some aspects, the frequency of the alternating electric field is 150 or 250 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 0.9 V/cm RMS. In some aspects, the alternating electric field has a field strength of any of those described herein.

### H. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of cholesterol lowering drugs and one or more materials for delivering alternating electric fields, such as the Optune system. For example disclosed are kits comprising one or more cholesterol lowering drugs, including, but not limited to, a hydroxymethylglutaryl-CoA (HMG CoA) reductase inhibitor, bempedoic acid, a fibrate, a resin, an apolipoprotein B synthesis inhibitor, a microsomal transfer protein inhibitor, PCSK9 inhibitors (Alirocumab (Praluent), Evolocumab (Repatha), Leqvio, LDL-C siRNA, L-IFTA+, BMS-962476, Pep2-8, Annexin A2, Pseurotin 1, Silibinin A, MK 0616, BMS 8444421, ISIS 394814, and SPC4061), a a fibric acid derivatives or fibrates (e.g. Tricor, Lopid, Trilipix, Lipofen, Antara, Riglide, Lofibra, Fibricor and Fenoglide), a cholesterol absorption inhibitor (e.g. Ezetimibe, (SCH 58235) and SCH-48461), Omega-3 fatty acids, or a citrate lyase inhibitor and one or more materials for delivering alternating electric fields, such as the Optune system.

### EXAMPLES

### A. Example 1

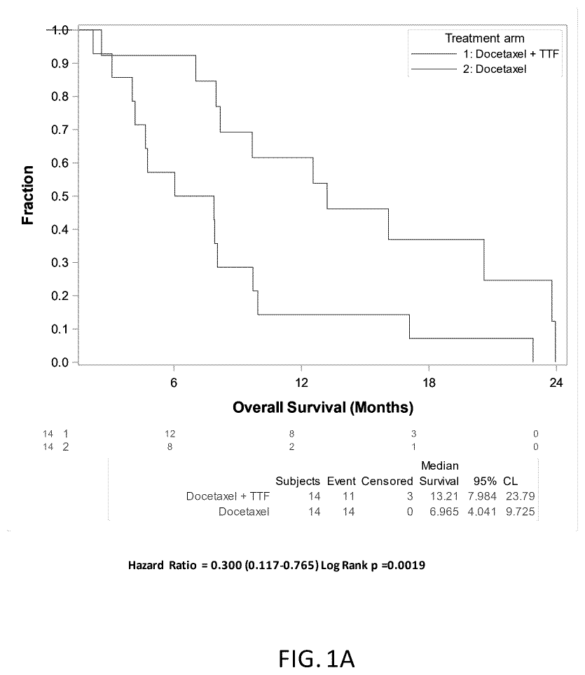

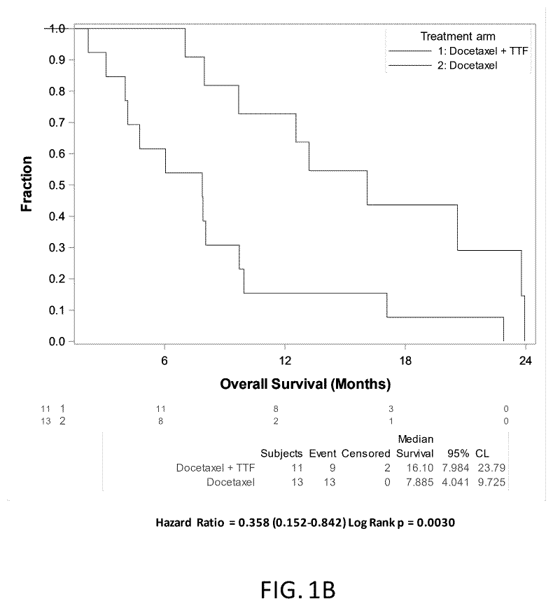

The use of statins in addition to TTFields treatment is associated with better overall survival in NSCLC patients compared to patients not receiving TTFields (FIG. 1). Repurposing cholesterol-lowering drugs (statins-HMG CoA reductase inhibitors, pcsk9 inhibitors or fibrates) concomitantly to TTFields can provide a better outcome in patients.

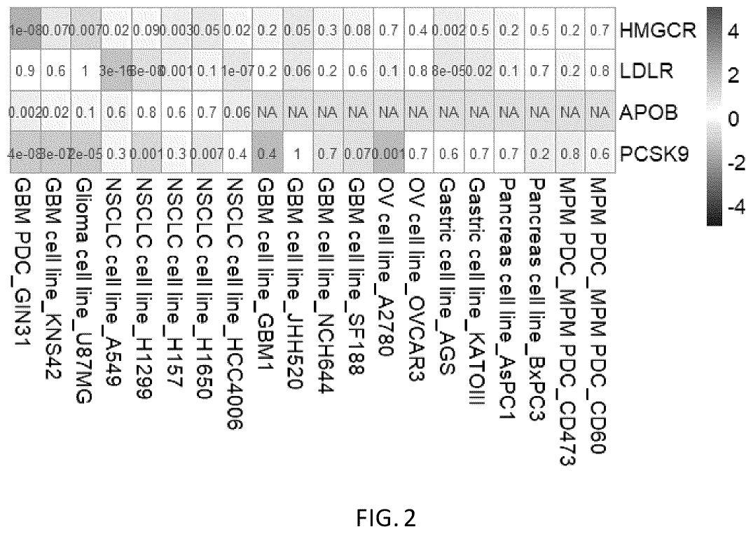

FIG. 2 shows genes associated with familial hypercholesterolemia which are partially upregulated following treatment with TTFields. An increase in HMGCR, LDLR, APOB and PCSK9 that are known to be overexpressed in cholesterol hypercholesterolemia is observed following 48 or 72 hours of TTFields application in cancer cell lines.

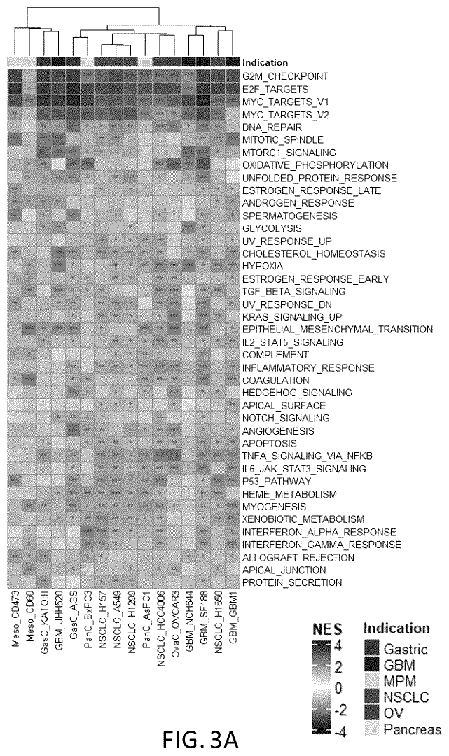

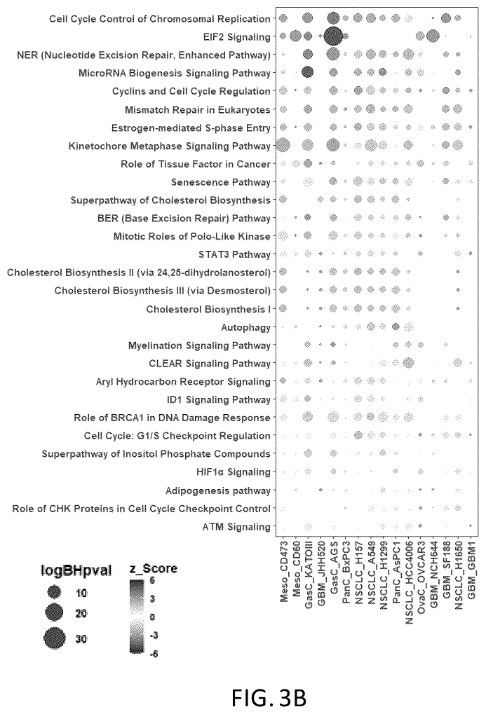

Results from a transcriptomic fingerprint of cancer cell response to Tumor Treating Fields (TTFields) (FIG. 3) showed that upregulated pathways across most datasets were associated with metabolism (mainly heme, cholesterol, and xenobiotics metabolism).

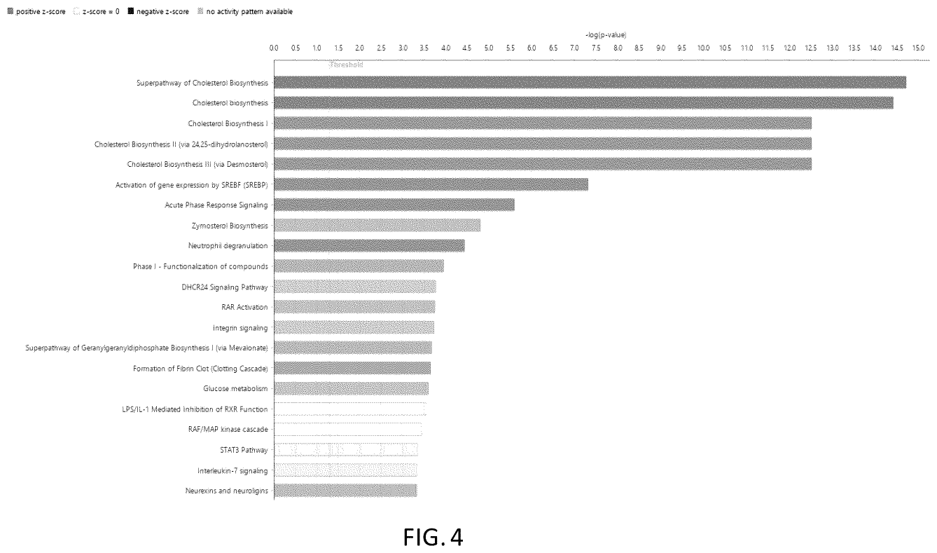

FIG. 4 shows hepatocellular carcinoma mice treated with TTFields upregulate pathways of cholesterol biosynthesis.

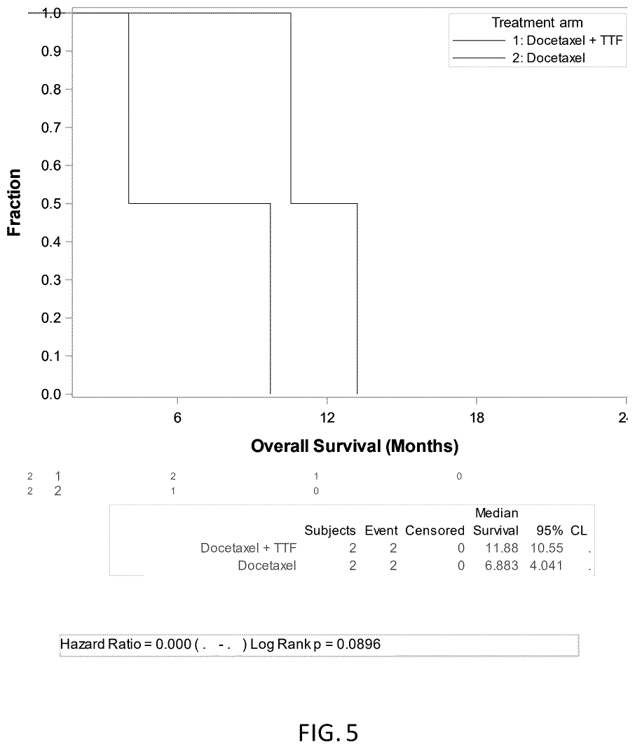

FIG. 5 shows an increase in median survival of patients receiving Simvastatin, Docetaxel and TTFields compared to patients receiving Simvastatin and Docetaxel.

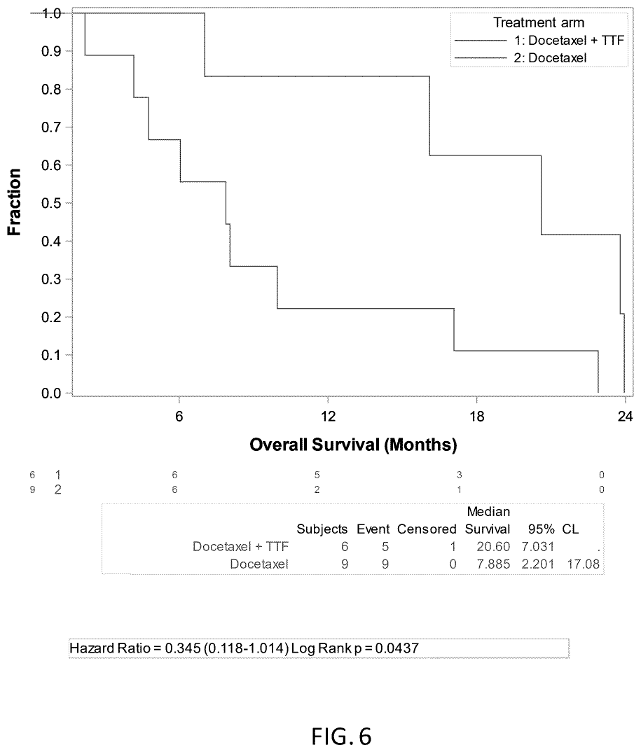

FIG. 6 shows an increase in median survival of patients receiving Atorvastatin, Docetaxel and TTFields compared to patients receiving Atorvastatin and Docetaxel.

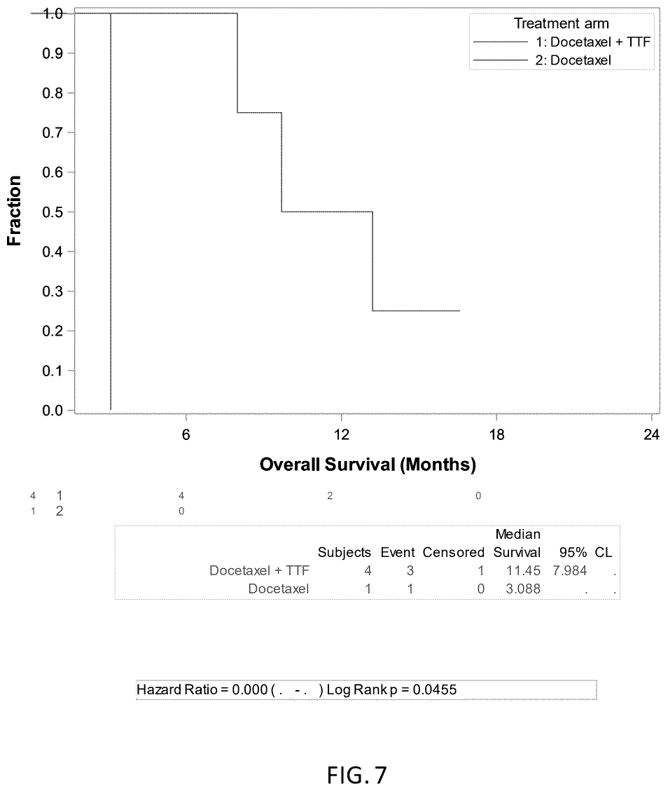

FIG. 7 shows an increase in median survival of patients receiving Rosuvastatin, Docetaxel and TTFields compared to patients receiving Rosuvastatin and Docetaxel.

### 1. Materials and Methods

TTFields application. TTFields were applied to cells using the Inovitro™ system.

Gastric/Pancreatic/Ovarian cancer dataset. Gastric AGS and KATOIII, pancreatic AsPC1 and BxPC3, and ovarian OVCAR3 human cell lines were obtained from the American Tissue Culture Collection (ATCC) and grown in appropriate growth media in a 37° C. humidified incubator supplied with 5% CO2. Cell growth media: AGS—F-12K media supplemented with 10% fetal bovine serum (FBS); KATOIII—IMDM media supplemented with 20% FBS; OVCAR3—RPMI1640 media supplemented with 20% FBS; AsPC1 and BxPC3—RPMI1640 media supplemented with 10% FBS. All media were further supplemented with 2 mM L-glutamine and 50 g/ml penicillin/streptomycin.

RNA was purified with TRI reagent as previously described, and analyzed with a 2100 bioanalyzer instrument (Agilent Technologies). RNA was quantified by Qubit (Thermo Fisher Scientific) and TapeStation (Agilent). For cDNA libraries preparation, the polyA fraction (mRNA) was purified, followed by fragmentation and generation of double-stranded cDNA. Agencourt AMPure XP beads (Beckman Coulter) were used for cleanup, followed by end repair, UMIs addition, adapter ligation and PCR amplification steps. Sequencing was done on Illumina NovoSeq 6000 system. Library generation and sequencing were done by the Crown Genomics institute of the Nancy and Stephen Grand Israel National Center for Personalized Medicine, Weizmann Institute of Science.

GBM dataset. GBM1, NCH644 and SF188 cells were cultured in DMEM media (Thermo Fisher) supplemented with 30% F-12 (Thermo Fisher), 2% B-27 (Thermo Fisher), 0.1% human EGF (Peprotech, AF-100-15), 0.1% FGF (Peprotech, 100-18B), 0.1% Heparin (Sigma, H0878), and 50 g/ml penicillin/streptomycin (Sigma Aldrich) to spontaneously form spheroids.

RNA was purified with TRI reagent as previously described, quantified using Qubit RNA HS Assay (Thermo Fisher Scientific), and quality measured by capillary electrophoresis using the FragmentAnalyzer and the DNF-471 Standard Sensitivity Assay (Agilent Technologies Inc., Santa Clara, USA). For cDNA library, preparation was performed by VAHTS Universal V6 RNA-seq Library Prep Kit (Illumina) according to the manufacturer's protocol, including mRNA capturing, fragmentation, cDNA synthesis, adapter ligation and library amplification. Bead purified libraries were normalized and sequenced on the HiSeq3000 system (Illumina).

Pleural mesothelioma and NSCLC datasets. Cell cultures and transcriptomic methods were described previously (for pleural mesothelioma, for NSCLC).

RNA quantification and differential expression. For RNA-seq samples (all but the NSCLC dataset) Fastq files were quality control screened with FASTQC [35]. Reads were aligned to the GENCODE human reference GRCh38.p13 with annotation file gencode.v36.annotation.gtf (both downloaded from https://www.gencodegenes.org/human/) using STAR-2.7.6a. Alignment and FastQC were assessed using multiqc. Bam files were indexed using samtools. Output files were organized into a sample/gene matrix. Normalization of reads into log Counts Per Million (CPM) was done by edgeR.

For the gastric/pancreatic/ovarian cancer dataset a pre-alignment step was taken, where UMIs from R2 were converted to the header using UMI_tools. Following STAR alignment and samtools indexing, files were deduplicated using UMI_tools. Reads per gene were counted with HTseq.

ComBat-seq was used to remove batch effect in cases where such an effect was introduced during sample preparation.

For the NSCLC dataset, normalized gene expression was used as provided in reference. The probe with the highest expression across all samples was taken as the representative probe per gene.

In all datasets outliers were screened using arrayQualityMetrics.

Differential expression was made for every TTFields treatment time point against control using Limma-voom; or Limma for the NSCLC dataset.

Data analysis and visualizations. Data analysis and visualizations were performed using R and Rstudio, unless specified otherwise. Packages for visualizations included pheatmap, ComplexHeatmap, ggplot2, heatmaply, seriation, and dendextend. Table and string organization were done using dplyr, tidyverse, tidyR, and stringr. Gene annotations were done with STRINGdb.

Gene set enrichment analysis. GSEA was performed on the gene lists preordered by their t score, using the GSEA software against the Hallmark genes set by the broad MsigDB and against the Reactome gene set.

Datasets Spearman paired correlation analysis. Correlation between log FC and between Normalized Gene set score was performed using corplot.

Active Pathways and enrichment map. P-values of the differential expression analysis were taken into ActivePathways, that calculated the Brown common p-value and then, through cytoscape, created an enrichment map for common significance pathways depicting cell line contribution to each pathway. The pathways were clustered according to the Reactome mother node classification using AutoAnnotate.

Canonical Pathways and Upstream Regulators Analysis. Ingenuity Canonical Pathways and Upstream Regulator Analysis was done using QIAGEN Ingenuity Pathway Analysis (IPA, https://digitalinsights.qiagen.com/IPA).

In some aspects, the materials and methods described in Leal et al. “Tumor Treating Fields therapy with standard systemic therapy versus standard systemic therapy alone in metastatic non-small-cell lung cancer following progression on or after platinum-based therapy (LUNAR): a randomised, open-label, pivotal phase 3 study” Lancet Oncol. 2023 September; 24(9):1002-1017 can be used, such methods and materials are hereby incorporated by reference in its entirety herein.

The disclosed experiments were based on a clinical trial in which subjects received Docetaxel or a combination of Docetaxel and TTFields. The patients also received statins during the clinical trial. The statins were not part of the clinical trial, however, the patients were previously on statins due to the chronic condition and continued to receive statins.

### B. Example 2—EF-11: GBM Patients with Hypertensive and Medications Duration from Start Treatment on Trial

In some aspects, the materials and methods described Stupp R et al. “NovoTTF-100A versus physician's choice chemotherapy in recurrent glioblastoma: a randomised phase III trial of a novel treatment modality”. Eur J Cancer. 2012 September; 48(14):2192-202. doi: 10.1016/j.ejca.2012.04.011. Epub 2012 May 18. PMID: 22608262. can be used, such methods and materials are hereby incorporated by reference in its entirety herein.

The disclosed experiments were based on a clinical trial in which GBM patients received best standard of care or TTFields. The patients also received statins during the clinical trial. The statins were not part of the clinical trial, however, the patients were previously on statins due to the chronic high cholesterol condition and continued to receive statins.

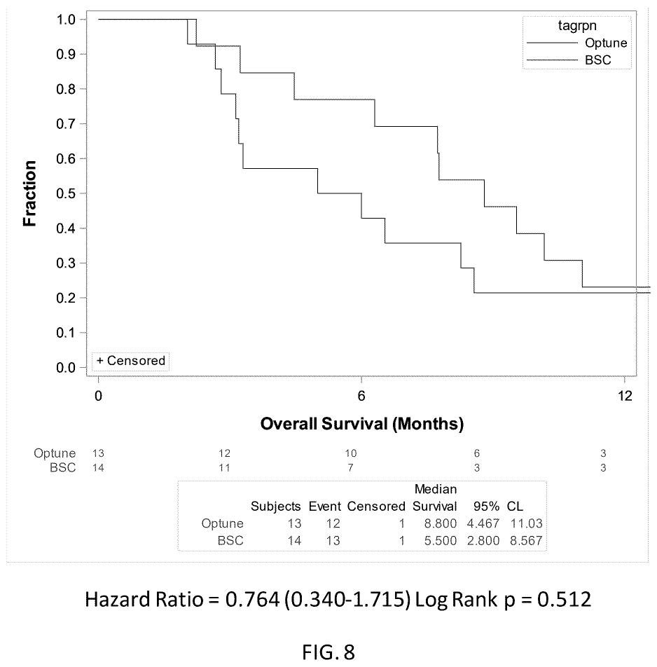

FIG. 8 shows the use of statins in addition to TTFields treatment is associated with better overall survival in GBM patients compared to patients not receiving TTFields (FIG. 8). Further supporting that repurposing cholesterol-lowering drugs (e.g., statins-HMG CoA reductase inhibitors, pcsk9 inhibitors or fibrates) concomitantly to TTFields can provide a better outcome in patients.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating a subject in need thereof comprising:
a) applying an alternating electric field to a target site of the subject in need thereof; and
b) administering one or more cholesterol lowering drugs to the subject in need thereof.

2. The method of claim 1, wherein the one or more cholesterol lowering drugs is one or more hydroxymethylglutaryl-CoA (HMG CoA) reductase inhibitors, bempedoic acid, fibrates, resins, apolipoprotein B synthesis inhibitors, microsomal transfer protein inhibitors, PCSK9 inhibitors, fibric acid derivatives or fibrates, cholesterol absorption inhibitors, LDL-C siRNA or citrate lyase inhibitors.

3. The method of claim 2, wherein the HMG CoA reductase inhibitor is atorvastatin (LIPITOR), fluvastatin (LESCOL XL), lovastatin (ALTOPREV), pitavastatin (LIVALO), pravastatin (PRAVACHOL), rosuvastatin (CRESTOR) or simvastatin (ZOCOR).

4. The method of claim 1, wherein the subject in need thereof has cancer.

5. The method of claim 1, wherein the cancer is lung cancer, hepatocellular cancer, brain cancer, pancreatic cancer, breast cancer, colon cancer, ovarian cancer, mesothelioma, renal cancer or glioblastoma.

6. The method of claim 1, wherein the subject has previously undergone chemotherapy.

7. The method of claim 1, wherein after applying alternating electric fields and before administering one or more cholesterol lowering drugs, detecting the presence of increased cholesterol and/or lipid biosynthesis in the subject and/or detecting the presence of upregulation of genes correlated with familial hypercholesterolemia in the subject.

8. The method of claim 1, wherein before applying alternating electric fields and before administering one or more cholesterol lowering drugs, detecting the presence of increased cholesterol and/or lipid biosynthesis in the subject and/or detecting the presence of upregulation of genes correlated with familial hypercholesterolemia in the subject.

9. The method of claim 1, wherein the target site comprises one or more cancer cells.

10. The method of claim 1, wherein the alternating electric fields are applied before, after, or simultaneously with administering the one or more cholesterol lowering drugs.

11. The method of claim 1, further comprising administering a chemotherapeutic agent to the subject.

12. The method of claim 11, wherein the chemotherapeutic agent is docetaxel.

13. The method of claim 1, wherein cancer cell proliferation is reduced.

14. The method of claim 13, wherein the cancer cell proliferation is reduced compared to subjects treated with docetaxel and statin alone.

15. The method of claim 1, wherein the alternating electric field has a frequency between 50 kHz and 1 MHz.

16. The method of claim 1, wherein the alternating electric field has a frequency of about 150 kHz or 200 kHz.

17. The method of claim 1, wherein the alternating electric field has a field strength of between 0.5 and 4 V/cm RMS.

18. The method of claim 1, wherein treatment with the alternating electric field is increased.

19. A method of increasing survival of a subject comprising:
a) applying an alternating electric field to a target site of the subject in need thereof; and
b) administering one or more cholesterol lowering drugs to the subject in need thereof.

20. A method of increasing efficacy of alternating electric fields in a cell comprising:
a) applying alternating electric fields to a cell; and
b) contacting one or more cholesterol lowering drugs to the cell,
thereby increasing efficacy of the alternating electric fields.
